(19) United States
(12) Patent Application Publication
(10) Pub. No.: US 2005/0075539 A1
(43) Pub. Date: Apr. 7, 2005

(54) Electronic endoscope

(76) Inventors: Dieter Schulz, Muhlheim a. D. (DE); Ralf Kiehn, Seitingen-Oberflacht (DE)

Correspondence Address: Douglas J. Christensen;Patterson, Thuente, Skaar & Christensen, P.A., 4800 IDS Center, 80 South Eighth Street, Minneapolis, MN, 55402, US

(21) Appl. No.: 10/959,818

(22) Filed: Oct. 6, 2004

(30) Foreign Application Priority Data
Oct. 7, 2003 (DE) 103 46 598.7

(51) Int. Cl. A61B 1/06; A61B 1/05

(52) U.S. Cl. 600/160; 600/178; 600/131

(57) ABSTRACT

An electronic endoscope is provided, comprising a shaft, an image-recording unit, which has an objective as well as, arranged following the objective, an image sensor for recording an object, and which is securely connected to the shaft at the distal end thereof, said endoscope further comprising a handpiece connected to the proximal end of the shaft, as well as an illumination device, connected to the shaft, for illuminating the object to be recorded, wherein the illumination device comprises at least one light emitting diode.

The invention relates to an electronic endoscope comprising a shaft, an image-recording unit, which has an objective as well as, arranged following the objective, an image sensor for recording an object, and which is securely connected to the shaft at the distal end thereof, said endoscope further comprising a handpiece connected to the proximal end of the shaft, as well as an illumination device connected to the shaft for illuminating the object to be recorded.

### BACKGROUND OF THE INVENTION

Such an electronic endoscope, which is described in DE 196 47 855 A1, for example, is employed, in particular, in the medical and technical fields. In many cases, the illumination device comprises a cold-light source whose light is coupled into a light conductor at the handpiece and is transmitted to the distal end of the shaft by the light conductor. This leads to a complicated and, thus, also expensive illumination device.

In view thereof, it is an object of the invention to improve the electronic endoscope of the aforementioned type such that it can be manufactured in a simple and inexpensive manner.

### SUMMARY OF THE INVENTION

According to the invention, this object is achieved in the afore-described electronic endoscope in that the illumination device comprises at least one light emitting diode as the light source. Nowadays, light emitting diodes are mass-produced articles which are extremely inexpensive and, on top of that, also have a very long life. This allows a simple structure of the electronic endoscope according to the invention, which additionally comprises an extremely inexpensive illumination device.

In a preferred embodiment, the endoscope according to the invention comprises, in particular in the handpiece, a control unit allowing pulsed operation of the at least one light emitting diode. This results in the advantage that the life of the light emitting diode can thus be further prolonged, while sufficient light for illuminating the object to be recorded is nevertheless provided by the at least one light emitting diode. Further, during pulsed operation, the light emitting diode may be operated with a stronger current, which advantageously allows to achieve a greater light intensity.

In particular, the control unit in the endoscope according to the invention may be provided such that the pulsed operation of the at least one light emitting diode is synchronized with the image-recording rate of the image sensor. This has the advantageous effect that the object to be illuminated is illuminated only during those time intervals in which it is recorded by means of the image sensor. Thus, excellent illumination is achieved while at the same time minimizing the energy requirement for the at least one light emitting diode.

In a further embodiment of the endoscope according to the invention, a power supply unit for the at least one light emitting diode and the image-recording unit is integrated in the endoscope, in particular in the handpiece. This leads to a very handy endoscope, in which the difficulties in handling caused by the power chords in conventional endoscopes no longer occur. The power supply unit may be fully integrated in the endoscope and not removable, or it may also be exchangeably connected to the endoscope. In particular, the power supply unit may comprise a battery, an accumulator, or a fuel cell. It is also possible for the power supply unit to include an inductively chargeable accumulator.

It is particularly preferred if the entire endoscope according to the invention, or only its shaft, is hermetically sealed in such a way that the endoscope, or the shaft, respectively, is autoclavable. Here, autoclavable means that the endoscope, or the shaft, respectively, can be exposed for at least several minutes to saturated water vapour of at least 130° C. for sterilization, without damaging the endoscope thereby (in particular, without allowing penetration of water vapour into the interior of the endoscope). In this case, the endoscope is very cost-effective for medical applications, because it can be optimally sterilized very quickly (by autoclaving) and can be used many times.

For hermetic sealing, according to a specific embodiment of the endoscope of the invention, the distal end of the shaft is hermetically sealed with a cover glass (the cover glass is preferably soldered to the shaft), with the objective and the at least one light emitting diode preferably being arranged at a distance from the cover glass. This ensures that, during autoclaving, no water vapour can penetrate into the interior space of the shaft incorporating the image-recording unit therein.

In particular, in the endoscope according to the invention, the at least one light emitting diode may be connected to the shaft and thermally insulated therefrom. This is particularly advantageous in case the at least one light emitting diode, according to its specification, can only be exposed to temperatures of less than 130° C. In this case, said thermal insulation is chosen such that, during autoclaving, the temperature of the light emitting diode does not exceed the specified maximum temperature although the shaft is heated to over 130° C. Said thermal insulation may be achieved, for example, by providing a material between the at least one light emitting diode and the shaft, said material having such a low thermal conductivity that the maximum temperature for the light emitting diode is just not achieved during autoclaving.

Of course, the image-recording unit or parts thereof may be thermally insulated, if required, relative to the shaft.

In a preferred embodiment of the endoscope according to the invention, the endoscope, and preferably the handpiece of the endoscope, has an image data processing unit arranged therein to which the signals from the image sensor are supplied. This enables processing of the signals already within the endoscope in such a manner that the desired image data are generated. In particular, desired manipulations of the image data, such as special filtering operations, rotations of the image position, etc., can be effected straight away.

Further, the endoscope, preferably the handpiece of the endoscope, may have a radio unit arranged therein for wireless transmission of the signals from the image sensor and/or the image data from the image data processing unit to a receiving unit, which is arranged separately and spaced apart from the endoscope. It is thus possible to provide an electronic endoscope which no longer requires any kind of cables (neither for power supply nor for image transmission), so that any hindrance caused by such cables during handling of the endoscope can be safely excluded.

The signals from the image sensor and/or the image data can be transmitted as digital or analog signals by the radio unit. In this case, the radio unit further includes, if necessary, an analog-digital converter or a digital-analog converter.

Further, the proximal end of the endoscope according to the invention may be provided with an image display device, which is connected to the endoscope and on which images recorded by the image-recording unit can be displayed. The image display device may be provided as an electronic display (e.g. a liquid crystal display, as in video cameras), so that the endoscope virtually has an electronic eyepiece. The electronic display may be provided, for example, as an LCoS display (Liquid Crystal on Silicon) or also as an LEP-S display (Light emitting Polymer on Silicon). In this case, the power supply unit provided in the endoscope preferably also serves as power supply source for the electronic display.

Further, the at least one light emitting diode may be arranged at the distal end of the shaft. This is particularly advantageous in that no light conductors are required, because the light emitting diode is provided directly in the area of the image-recording unit.

In particular, a plurality of light emitting diodes is provided, which surround the objective, when viewing the objective from above (i.e., along the optical axis of the objective). This allows to achieve a very uniform illumination of the object to be recorded.

If a plurality of light emitting diodes is provided, pulsed operation of said light emitting diodes may be effected in groups or individually, in an alternating manner, during pulsed operation of said light emitting diodes. It is also possible to adjust the brightness of the illumination by the number of simultaneously switched-on light emitting diodes.

The at least one light emitting diode is preferably a white light emitting diode, i.e. a light emitting diode which emits white light. Of course, use may also be made of a light emitting diode which emits colored light. It is further possible to provide light emitting diodes emitting light of different colors, the light from said light emitting diodes resulting in a predetermined color or in white light.

The endoscope or the shaft, respectively, may be flexible, semi-flexible or rigid.

The invention will be explained in more detail below, by way of example, with reference to an embodiment example and to the attached drawings, wherein:

### DESCRIPTION OF THE DRAWINGS

FIG. 1 shows a lateral view of the embodiment of the electronic endoscope, and

FIG. 2 shows a view of the distal end of the shaft of the endoscope shown in FIG. 1.

### DETAILED DESCRIPTION OF PREFERRED EMBODIMENTS

The autoclavable endoscope shown in the Figures is intended, in particular, for use in the medical field and comprises a shaft 1 having an outer tube 2 and an inner tube 3, which is arranged concentrically to the former. The proximal end of the shaft (and, thus, of both tubes 2 and 3) is connected to a handpiece 4 in a hermetically sealed manner, while in the inner tube 3 of the shaft 1, at the distal end of said shaft, an image-recording unit 5 is arranged, which comprises an objective 6 and an image sensor 7 arranged following the objective 6. The image-recording unit 5 is preferably securely connected to the shaft 1, so that the image-recording unit 5 is arranged in the shaft 1 in a stationary manner. However, the objective may certainly be provided as a zoom objective, for example. The image sensor 7 is preferably a CCD or CMOS image sensor.

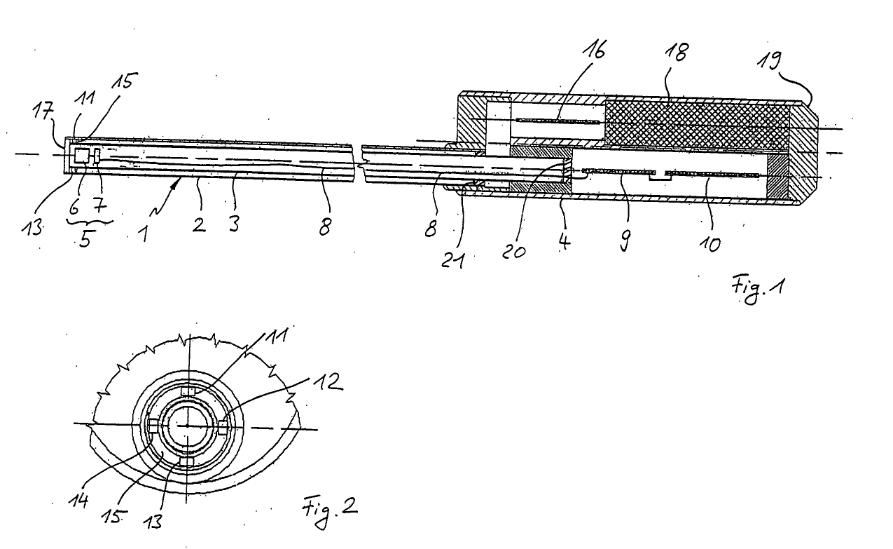

The image sensor 7 is connected, via an electronic cable connection 8 schematically indicated in FIG. 1, with an image data processing unit 9 arranged in the handpiece 4. The image data processing unit 9 processes the signals from the image sensor 7 to image data and transmits them via a radio module 10 to a receiver, not shown, which is spaced apart and separated from the endoscope. Said receiver may be a conventional computer comprising a screen and a corresponding radio reception module, with the computer displaying the transmitted image signals as an image on said screen.

Further, four light emitting diodes 11, 12, 13, and 14 are arranged at the distal end of the shaft, between the outer and inner tubes 2, 3. In the embodiment example described herein, the light emitting diodes 11 to 14 are fitted onto an annular carrier 15, which fits exactly between the outer and inner tubes 2, 3. The light emitting diodes 11 to 14 are connected, via a cable connection (not shown) extending between the outer and inner tubes 2, 3, to a control unit 16 provided in the handpiece 4. The light emitting diodes 11 to 14 are preferably white light emitting diodes. It is also possible, of course, to provide light emitting diodes 11 to 14 of different colors according to the particular case of application.

The distal end of the shaft 1 is hermetically sealed by a cover glass 17.

Further, an internal power supply (power supply unit) 18 is arranged in the handpiece 4, which may also be provided as a pistol grip. The power supply unit 18, which is an inductively chargeable accumulator in the presently described example, serves to supply power to the image sensor 7 as well as the control unit 16, the image data processing unit 9 and the radio module 10. The proximal end of the handpiece 4 is hermetically sealed by a cover 19.

Alternatively, it is also possible that the endoscope shaft 1 is releasably connected to the handpiece 4 and comprises a seal 20 for the inner tube and a seal 21 for the space between the inner and outer tubes 2, 3, at the proximal end of the shaft 1, so that the entire shaft 1 is autoclavable. In this case, the electrical connections to the image data processing unit 8 and to the control unit 16 certainly need to be releasable.

The control unit 16 serves to operate the light emitting diodes 11 to 14 in a pulsed manner, said pulsed operation being synchronized with the image-recording repetition rate of the image sensor 7 so that the object to be recorded is illuminated only when an image is actually being recorded by the image recording means 5.

Of course, the power supply unit 18 may also be exchangeably provided. In this case, the power supply unit 18 may comprise a battery, an accumulator, or a fuel cell.

Further, it is also possible for the cable connection 8 to be replaced by a wireless link. The same goes for the connection between the control unit 16, on the one hand, and the light emitting diodes 11 to 14, on the other hand.

It is also possible to provide the connection between the power supply unit 18, on the one hand, and the light emitting diodes 11 to 14 and the image-recording unit 5 as a wireless link, e.g. to replace it by an inductive coupling.

1. An electronic endoscope comprising a shaft, an image-recording unit, which has an objective as well as, arranged following the objective, an image sensor for recording an object, and which is securely connected to the shaft at the distal end thereof, said endoscope further comprising a handpiece connected to the proximal end of the shaft, as well as an illumination device, connected to the shaft, for illuminating the object to be recorded, wherein the illumination device comprises at least one light emitting diode.

2. The endoscope as claimed in claim 1, wherein the endoscope, in particular the handpiece, has a control unit arranged therein allowing pulsed operation of the at least one light emitting diode.

3. The endoscope as claimed in claim 2, wherein the pulsed operation of the at least one light emitting diode is synchronized with the image-recording rate of the image sensor.

4. The endoscope as claimed in claim 1, wherein the endoscope, in particular the handpiece, has a power supply unit incorporated therein for the at least one light emitting diode and the image-recording unit.

5. The endoscope as claimed in claim 1, wherein the endoscope is hermetically sealed in such a manner that it is autoclavable.

6. The endoscope as claimed in claim 1, wherein the shaft is hermetically sealed in such a manner that the shaft is autoclavable.

7. The endoscope as claimed in 5, wherein the distal end of the shaft is hermetically sealed by a cover glass, and the objective and the at least one light emitting diode are spaced apart from the cover glass.

8. The endoscope as claimed in 6, wherein the distal end of the shaft is hermetically sealed by a cover glass, and the objective and the at least one light emitting diode are spaced apart from the cover glass.

9. The endoscope as claimed in claim 1, wherein the at least one light emitting diode is connected to the shaft and thermally insulated therefrom.

10. The endoscope as claimed in claim 1, wherein the endoscope, and preferably the handpiece, has an image data processing unit arranged therein to which the signals from the image sensor are supplied.

11. The endoscope as claimed in claim 1, wherein a radio unit is provided which is preferably arranged in the handpiece, said radio unit transmitting the signals from the image sensor or the image data from the image data processing unit in a wireless manner to a receiving unit which is spaced apart and separated from the endoscope.

12. The endoscope as claimed in claim 1, wherein, at the proximal end of the endoscope, an image display device is provided on which the recorded images are displayable.

13. The endoscope as claimed in claim 1, wherein the at least one light emitting diode is arranged at the distal end of the shaft.

14. The endoscope as claimed in claim 13, wherein a plurality of light emitting diodes are arranged at the distal end of the shaft in such a manner that, when the objective is viewed from above, they surround the objective.

15. The endoscope as claimed in claim 1, wherein the at least one light emitting diode is a white light emitting diode.

16. The endoscope as claimed in claim 1, wherein the shaft is flexible.

17. The endoscope as claimed in claim 1, wherein the shaft is rigid.

18. An electronic endoscope comprising a shaft, an image-recording unit, which has an objective as well as, arranged following the objective, an image sensor for recording an object, and which is securely connected to the shaft at the distal end thereof, said endoscope further comprising a handpiece connected to the proximal end of the shaft, as well as an illumination device, connected to the shaft, for illuminating the object to be recorded, wherein the illumination device comprises at least one light emitting diode and wherein the at least one light emitting diode is connected to the shaft and thermally insulated therefrom.
